(19) United States
(12) Patent Application Publication
(10) Pub. No.: US 2026/0193646 A1
(43) Pub. Date: Jul. 9, 2026

(54) Fluorinated Phenylalanine Ma PylRS/tRNA Pair GCE Toolkit

(71) Applicant: Oregon State University, Corvallis, OR (US)

(72) Inventors: Ryan A. Mehl, Corvallis, OR (US); Richard B. Cooley, Corvallis, OR (US); Nathan D. Alexander, Corvallis, OR (US)

(73) Assignee: Oregon State University, Corvallis, OR (US)

(21) Appl. No.: 19/445,258

(22) Filed: Jan. 9, 2026

Related U.S. Application Data
(60) Provisional application No. 63/743,633, filed on Jan. 9, 2025.
(60) Provisional application No. 63/744,036, filed on Jan. 10, 2025.

(51) Int. Cl. C12N 15/11; C12N 9/00

(52) U.S. Cl. CPC C12N 15/111; C12N 9/93; C12Y 601/01026

(57) ABSTRACT

Pyrrolysine-based aminoacyl-tRNA synthetases competent to recognize a fluorinated phenylalanine analog and aminoacylate a tRNA; pyrrolysine-based aminoacyl-tRNA synthetase/tRNA pairs comprising the pyrrolysine-based aminoacyl-tRNA synthetase and a tRNA competent for site-specific encoding of a fluorinated phenylalanine analog within a protein; methods of site-specific encoding of a fluorinated phenylalanine analog in a cell, comprising transfecting/transforming/transducing a cell with the pyrrolysine-based aminoacyl-tRNA synthetase and the tRNA competent for site-specific encoding; and kits for in cellulo production of a fluorinated phenylalanine-labeled protein using DNA encoding the pyrrolysine-based aminoacyl-tRNA synthetase and the tRNA competent for site-specific encoding of a fluorinated phenylalanine analog, and a fluorinated phenylalanine analog.

### CROSS-REFERENCES TO RELATED APPLICATIONS

This application claims the benefit of U.S. Patent Application No. 63/743,633, filed Jan. 9, 2025, and U.S. Patent Application No. 63/744,036, filed Jan. 10, 2025, the disclosure of each of which is expressly incorporated herein by reference in its entirety

### STATEMENT OF GOVERNMENT LICENSE RIGHTS

This invention was made with Government support under RM1 GM144227 awarded by the National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3014-P46US_Seq_List_20260107.xml. The XML file is 41,525 bytes; was created on Jan. 7, 2026; and is being submitted electronically via Patent Center with the filing of the specification.

### BACKGROUND

Solution-state nuclear magnetic resonance (NMR) spectroscopy provides atomic-resolution structure and dynamics information of macromolecules under physiological conditions. In systems with a total molecular mass greater than 35 kDa fast relaxation of NMR active nuclei leads to resonance broadening and reduction in signal intensity. In addition, the concomitant increase in resonance number within a limited spectral space results in a high degree of signal overlap. This overlap can be addressed using specific isotopic labelling techniques. The ¹⁹F nucleus is NMR active, has a 100% natural abundance, and a chemical shift that is very sensitive to its electronic environment. These advantageous properties have made ¹⁹F a highly sought-after probe in the study of biomolecules, including GPCRs, viral capsids, and enzyme catalysis, as well as in drug discovery campaigns. ¹⁹F probes have been incorporated into proteins through two primary methods: by labelling aromatic amino acids during recombinant protein expression, or by attaching CF₃³ or CFD₂-containing moieties to cysteine residues via chemical reactions on purified proteins.

Genetic code expansion (GCE) offers an ideal strategy to site-specifically encode a fluorinated phenylalanine (e.g., 4-¹⁹F Phe) into a protein. However, the structural similarities between 4-¹⁹F Phe and natural Phe pose substantial challenges when developing translational systems to selectively encode it. Although GCE systems that install multiply-substituted fluorinated phenylalanine derivatives have been reported, no such in-cell system exists to encode mono-substituted fluorinated Phe amino acids.

A need exists for a GCE system for site-specifically encoding a fluorinated phenylalanine into a protein. The present invention seeks to fulfill this need and provides further related advantages.

### SUMMARY

The present disclosure provides a family of novel, recombinant aminoacyl tRNA synthetase enzymes that are useful in making recombinant proteins that include at least one fluorinated phenylalanine amino acid. Thus, in addition to the novel recombinant aminoacyl tRNA enzymes, the present invention provides recombinant proteins that incorporate a site-specifically incorporated fluorinated phenylalanine amino acid. In some embodiments, the fluorinated phenylalanine containing recombinant protein is a therapeutic protein.

The recombinant aminoacyl tRNA synthetase enzymes may be co-expressed in a cell with a corresponding tRNA to enable the robust, site-specific, scalable encoding of multiple different types of fluorinated phenylalanine amino acids in recombinant proteins expressed in mammalian cell lines and bacteria. The recombinant aminoacyl tRNA synthetase enzyme and a corresponding tRNA may also be used in a cell free system to produce recombinant proteins that incorporate a fluorinated phenylalanine amino acid.

The present disclosure provides in certain embodiments, a novel pyrrolysine-based aminoacyl-tRNA synthetase (RS) paired with a tRNA to form a pyrrolysine-based aminoacyl-tRNA synthetase/tRNA (RS/tRNA or “PYLRS/TRNA”) pair.

### DETAILED DESCRIPTION

The present disclosure provides pyrrolysine-based aminoacyl-tRNA synthetases competent to recognize a fluorinated phenylalanine analog and aminoacylate a tRNA; pyrrolysine-based aminoacyl-tRNA synthetase/tRNA pairs comprising the pyrrolysine-based aminoacyl-tRNA synthetase and a tRNA competent for site-specific encoding of a fluorinated phenylalanine analog within a protein; methods of site-specific encoding of a fluorinated phenylalanine analog in a cell, comprising transfecting/transforming/transducing a cell with the pyrrolysine-based aminoacyl-tRNA synthetase and the tRNA competent for site-specific encoding; and kits for in cellulo production of a fluorinated phenylalanine-labeled protein using DNA encoding the pyrrolysine-based aminoacyl-tRNA synthetase and the tRNA competent for site-specific encoding of a fluorinated phenylalanine analog, and a fluorinated phenylalanine analog.

In one aspect, the present disclosure provides pyrrolysine-based aminoacyl-tRNA synthetases for use with their corresponding tRNA to incorporate a fluorinated phenylalanine analog into proteins of choice with site specificity.

In certain embodiments, the pyrrolysine-based aminoacyl-tRNA synthetase competent to recognize a fluorinated phenylalanine analog and aminoacylate a tRNA is selected from the following pyrrolysine-based aminoacyl-tRNA synthetases, characterized by their designated amino acid sequence:
2345D2 (SEQ ID NO:3);
2345E3 (SEQ ID NO:4);
2345E4 (SEQ ID NO:5);
2345E6 (SEQ ID NO:6);
236C9 (SEQ ID NO:7);
236E9 (SEQ ID NO:8);
236F7 (SEQ ID NO:9);
236G11 (SEQ ID NO:10);
236G7 (SEQ ID NO:11);
24B3 (SEQ ID NO:12);
24D6 (SEQ ID NO:13);
24G1 (SEQ ID NO:14);
24G5 (SEQ ID NO:15);
25D8 (SEQ ID NO:16);
25E7 (SEQ ID NO:17);
25F7 (SEQ ID NO:18);
25H10 (SEQ ID NO:19);
26B2 (SEQ ID NO:20);
26B3 (SEQ ID NO:21);
26B4 (SEQ ID NO:22);
26C6 (SEQ ID NO:23);
26D5 (SEQ ID NO:24);
2B5 (SEQ ID NO:25);
2C3 (SEQ ID NO:26);
2C5 (SEQ ID NO:27);
2D1 (SEQ ID NO:28);
4A7 (SEQ ID NO:29);
4C8 (SEQ ID NO:30);
penB7 (SEQ ID NO:31);
penF10 (SEQ ID NO:32);
penF7 (SEQ ID NO:33);
tpG2 (SEQ ID NO:34);
tpH6 (SEQ ID NO:35); and
2345C3 (SEQ ID NO:36).

The synthetases described herein are Methanomethylophilus alvus pyrrolysine-based aminoacyl-tRNA synthetases (MaRSs or MA PYLRSs) engineered for use with their corresponding tRNA to incorporate a fluorinated phenylalanine analog with site specificity into proteins of choice. The tRNA synthetases are listed below and characterized by their amino acid mutations:
2345D2 (SEQ ID NO:3): L125I, N166H, V168T, A223K, Y239T;
2345E3 (SEQ ID NO:4): L125I, N166H, V168S, A223K, Y239T;
2345E4 (SEQ ID NO:5): L125V, N166H, V168Q, A223G, Y239W;
2345E6 (SEQ ID NO:6): L125E, N166H, V168Q, A223S, Y239W;
236C9 (SEQ ID NO:7): L125I, N166Q, V168S, A223A, Y239W;
236E9 (SEQ ID NO:8): L125L, N166A, V168I, A223A, Y239T;
236F7 (SEQ ID NO:9): L125T, N166T, V168I, A223A, Y239W;
236G11 (SEQ ID NO:10): L125I, N166A, V168C, A223A, Y239W;
236G7 (SEQ ID NO:11): L125I, N166C, V168M, A223A, Y239A;
24B3 (SEQ ID NO:12): L125H, N166C, V168Y, A223S, Y239W;
24D6 (SEQ ID NO:13): L125A, N166T, V168Y, A223A, Y239T;
24G1 (SEQ ID NO:14): L125L, N166Q, V168S, A223A, Y239W;
24G5 (SEQ ID NO:15): L125L, N166H, V168H, A223S, Y239W;
25D8 (SEQ ID NO:16): L125C, N166H, V168Q, A223C, Y239W;
25E7 (SEQ ID NO:17): L125L, N166C, V168K, A223T, Y239W;
25F7 (SEQ ID NO:18): L125H, N166Q, V168I, A223A, Y239W;
25H10 (SEQ ID NO:19): L125I, N166A, V168K, A223A, Y239W;
26B2 (SEQ ID NO:20): L125I, N166A, V168M, A223A, Y239W;
26B3 (SEQ ID NO:21): L125L, N166A, V168H, A223A, H227I, Y228P, Y239W;
26B4 (SEQ ID NO:22): L125L, N166A, V168Q, A223A, Y239W;
26C6 (SEQ ID NO:23): L125L, N166V, V168Y, A223A, Y239T;
26D5 (SEQ ID NO:24): L125L, N166A, V168F, A223G, Y239W;
2B5 (SEQ ID NO:25): L125Q, N166Q, V168S, A223A, Y239W;
2C3 (SEQ ID NO:26): L125A, N166L, V168K, A223A, Y239W;
2C5 (SEQ ID NO:27): L125V, N166Q, V168T, A223A, Y239W;
2D1 (SEQ ID NO:28): L125A, N166Q, V168T, A223A, Y239W;
4A7 (SEQ ID NO:29): L125M, N166H, V168H, A223K, Y239T;
4C8 (SEQ ID NO:30): L125L, N166C, V168Y, A223K, Y239T;
penB7 (SEQ ID NO:31): L125F, N166M, V168F, A223G, Y239W;
penF10 (SEQ ID NO:32): L125L, N166V, V168K, A223G, Y239W;
penF7 (SEQ ID NO:33): L125V, N166C, V168K, A223G, Y239W;
tpG2 (SEQ ID NO:34): L125L, N166Q, V168T, A223S, Y239W;
tpH6 (SEQ ID NO:35): L125I, N166Q, V168V, A223A, Y239W; and
2345C3 (SEQ ID NO:36): L125V, N166H, V168Q, A223S, Y239W.

In a related aspect, the disclosure provides synthetase/tRNA pairs for incorporating a fluorinated phenylalanine analog with site specificity into a protein of choice.

In certain embodiments, the disclosure provides a pyrrolysine-based aminoacyl-tRNA synthetase/tRNA pair comprising a pyrrolysine-based aminoacyl-tRNA synthetase as described herein and a tRNA competent for site-specific encoding of a fluorinated phenylalanine analog within a protein.

In certain of these embodiments, the tRNA useful in the methods described herein using prokaryotic cells is encoded by DNA having SEQ ID NO: 1.

In other of these embodiments, the tRNA useful in the methods described herein using eukaryotic cells is encoded by DNA having SEQ ID NO: 2.

As used herein, the term “pyrrolysine-based aminoacyl-tRNA synthetase/tRNA pair” competent for site-specific encoding of a fluorinated phenylalanine analog within a protein is a synthetase (RS) that provides either (i) a 5-fold protein expression with the fluorinated phenylalanine analog in the media vs protein expression without the fluorinated phenylalanine analog or (ii) mass spectrometric analysis of the resultant protein shows about 80% or about 90% site-specific encoding of the fluorinated phenylalanine analog.

In a further aspect, the disclosure provides methods for using the pyrrolysine-based aminoacyl-tRNA synthetase/tRNA pair.

In certain embodiments, the disclosure provides a method of site-specific encoding of a fluorinated phenylalanine analog in a cell, comprising transfecting/transforming/transducing a cell with a pyrrolysine-based aminoacyl-tRNA synthetase described herein and a tRNA competent for site-specific encoding of a fluorinated phenylalanine analog within a protein to provide a transfected cell, and contacting the transfected cell with a fluorinated phenylalanine analog to provide a protein site-specifically encoded with a fluorinated phenylalanine analog.

In related embodiments, the disclosure provides an in cellulo method for genetically encoding a protein or a polypeptide of interest, comprising incorporating a fluorinated phenylalanine analog into a protein by genetic encoding using the pyrrolysine-based aminoacyl-tRNA synthetase of claim 1 and a tRNA competent for site-specific encoding of a fluorinated phenylalanine analog within a protein.

In certain embodiments of the methods, the cell is a prokaryotic cell (e.g., an E. coli cell). In other embodiments of the methods, the cell is a eukaryotic cell (e.g., a mammalian cell).

The compositions and methods described herein are useful for site-specific incorporation of a fluorinated phenylalanine analog into a protein. Representative fluorinated phenylalanine analogs that are advantageously incorporated include (a) 2-fluorophenylalanine (or 2-monofluorophenylalanine), (b) 4-fluorophenylalanine (or 4mono-fluorophenylalanine), (c) 2,4-difluorophenylalanine, (d) 2,5-difluorophenylalanine, (e) 2,6-difluorophenylalanine, (f) 2,3,6-trifluorophenylalanine, (g) 2,3,4,5-tetrafluorophenylalanine, and (h) pentafluorophenylalanine. The chemical structures of these fluorinated phenylalanine analogs are shown below.

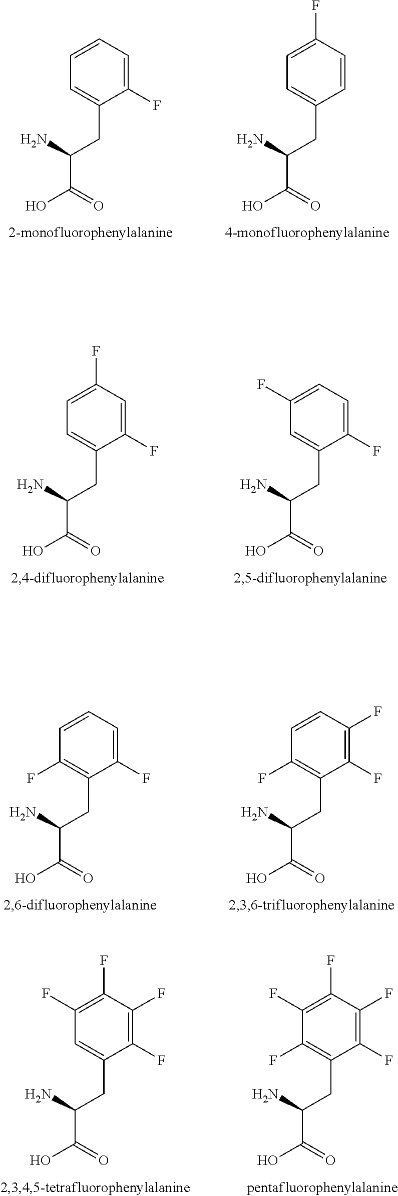

The present disclosure provides for the effective incorporation of fluorinated phenylalanine analogs into proteins using the pyrrolysine-based aminoacyl-tRNA synthetases and a competent tRNAs.

2,3,4,5-Tetrafluorophenylalanine has been site-specifically incorporated into Superfolder GFP (sfGFP) in prokaryotic cells using synthetases 2345D2 (SEQ ID NO: 3), 2345E3 (SEQ ID NO:4), 2345E4 (SEQ ID NO:5), 2345E6 (SEQ ID NO:6), and 2345C3 (SEQ ID NO:36), and tRNA encoded by DNA having SEQ ID NO: 1. This fluorinated phenylalanine analog has also been site-specifically incorporated into sfGFP in eukaryotic cells using synthetase 2345D2 (SEQ ID NO:3) and tRNA encoded by DNA having SEQ ID NO: 2.

2,3,6-Trifluorophenylalanine has been site-specifically incorporated into Superfolder GFP (sfGFP) in prokaryotic cells using synthetases 236C9 (SEQ ID NO:7), 236E9 (SEQ ID NO:8), 236F7 (SEQ ID NO:9), 236G11 (SEQ ID NO:10), and 236G7 (SEQ ID NO:11), and tRNA encoded by DNA having SEQ ID NO: 1.

2,4-Difluorophenylalanine has been site-specifically incorporated into Superfolder GFP (sfGFP) in prokaryotic cells using synthetases 24B3 (SEQ ID NO:12), 24D6 (SEQ ID NO:13), 24G1 (SEQ ID NO:14), and 24G5 (SEQ ID NO:15), and tRNA encoded by DNA having SEQ ID NO: 1. This fluorinated phenylalanine analog has also been site-specifically incorporated into sfGFP in eukaryotic cells using synthetase 24G1 (SEQ ID NO:14) and tRNA encoded by DNA having SEQ ID NO: 2.

2,5-Difluorophenylalanine has been site-specifically incorporated into Superfolder GFP (sfGFP) in prokaryotic cells using synthetases 25D8 (SEQ ID NO:16), 25E7 (SEQ ID NO:17), 25F7 (SEQ ID NO:18), and 25H10 (SEQ ID NO:19), and tRNA encoded by DNA having SEQ ID NO: 1.

2,6-Difluorophenylalanine has been site-specifically incorporated into Superfolder GFP (sfGFP) in prokaryotic cells using synthetases 26B2 (SEQ ID NO:20), 26B3 (SEQ ID NO:21), 26B4 (SEQ ID NO:22), 26C6 (SEQ ID NO:23), and 26D5 (SEQ ID NO: 23), and tRNA encoded by DNA having SEQ ID NO: 1. This fluorinated phenylalanine analog has also been site-specifically incorporated into sfGFP in eukaryotic cells using synthetase 26D5 (SEQ ID NO:24) and tRNA encoded by DNA having SEQ ID NO: 2.

2-Fluorophenylalanine has been site-specifically incorporated into Superfolder GFP (sfGFP) in prokaryotic cells using synthetases 2B5 (SEQ ID NO:25), 2C3 (SEQ ID NO:26), 2C5 (SEQ ID NO:27), and 2D1 (SEQ ID NO:28), and tRNA encoded by DNA having SEQ ID NO: 1.

4-Fluorophenylalanine has been site-specifically incorporated into Superfolder GFP (sfGFP) in prokaryotic cells and eukaryotic cells using synthetases 4A7 (SEQ ID NO:29) and 4C8 (SEQ ID NO:30), and tRNA encoded by DNA having SEQ ID NO: 1 and tRNA encoded by DNA having SEQ ID NO:2, respectively.

Pentafluorophenylalanine has been site-specifically incorporated into Superfolder GFP (sfGFP) in prokaryotic cells using synthetases penB7 (SEQ ID NO:31), penF10 (SEQ ID NO:32), penF7 (SEQ ID NO:33), and tpG2 (SEQ ID NO:34), and tRNA encoded by DNA having SEQ ID NO: 1 in prokaryotic cells. This fluorinated phenylalanine analog has also been site-specifically incorporated into sfGFP in eukaryotic cells using synthetase penF7 (SEQ ID NO:33) and tRNA encoded by DNA having SEQ ID NO: 2.

2,3,6-Trifluorophenylalanine has been site-specifically incorporated into Superfolder GFP (sfGFP) in prokaryotic cells and eukaryotic cells using synthetase tpH6 (SEQ ID NO:35), and tRNA encoded by DNA having SEQ ID NO: 1 and tRNA encoded by DNA having SEQ ID NO:2, respectively.

In another aspect, the disclosure provides kits for in cellulo production of a fluorinated phenylalanine-labeled protein using the compositions and methods described herein.

In certain embodiments, the kit for in cellulo production of a fluorinated phenylalanine-labeled protein, comprises (a) DNA encoding a pyrrolysine-based aminoacyl-tRNA synthetase as described herein and a tRNA competent for site-specific encoding of a fluorinated phenylalanine analog; and (b) a fluorinated phenylalanine analog as described herein.

The kit described includes DNA encoding a pyrrolysine-based aminoacyl-tRNA synthetase and a tRNA competent for site-specific encoding of a fluorinated phenylalanine analog. In certain embodiments, the DNA encoding the synthetase and tRNA is a plasmid that interacts with the genome of the host cell and facilitates production of the synthetase and tRNA.

Methods for General Use of FluoroPhe MaPyIRS/tRNA Pairs to Encode a Fluorinated Phe Analog into a Protein of Interest
Expression of F-Phe-sfGFP in Escherichia coli

Expression sfGFP-150FPhe for each analog using a respective RS/tRNA pair was done in both (A) DH10B by co-transforming cells with a plasmid encoding the F Phe RS and a plasmid encoding each an arabinose inducible sfGFP-N150TAG gene and tRNA^{Pyl} and (B) BL21(DE3) by co-transforming cells with a plasmid encoding the F Phe RS and tRNA^{Pyl} and a plasmid encoding a lactose inducible sfGFP-N150TAG gene. Transformants were spread onto an LB agar plate and incubated at 37° C. for 16 h. For (A) 1 colony or (B) 50-200 colonies were transferred 5 mL non induction media and incubated for at 37° C. with shaking at 250 rpm for 16 h (Studier, F. W. Protein Production by Auto-Induction in High Density Shaking Cultures. Protein Expr. Purif. 2005, 41 (1), 207-234). Each F Phe analog was prepared as a stock solution using DI water and an equimolar concentration of NaOH. Autoinduction media cultures with (A) arabinose or (B) lactose were inoculated by adding 5% v/v transformant culture and supplemented with 0.1-1.0 mM F Phe analog. Cultures were grown 20-24 h and cells were collected. Protein was purified by first lysing cells in lysis buffer containing 50 mM sodium phosphate, 500 mM NaCl, 5 mM imidazole (pH 7.4). Lysate was clarified by centrifuge at 28,000 rcf then applied to HisPur™ cobalt resin equilibrated with lysis buffer. Protein was eluted using elution buffer containing 50 mM sodium phosphate, 500 mM NaCl, 150 mM imidazole (pH 7.4) and desalted into buffer containing 50 mM ammonium bicarbonate.

Mammalian Optimization of tfmW-Protein Expression

Before transfection, HEK-293T cells were seeded at about 40% confluency in 24-well plates containing fresh Corning DMEM (Dulbecco's Modified Eagle's Medium) containing 10% FBS and grown to about 70-80% confluency. Transfections were performed using 600 ng total DNA per well, following the manufacturer's instructions for JetPrime reagent (PolyPlus, VWR, USA). A 100 mM stock solution of each F Phe was prepared in water by adding one equimolar equivalent of NaOH, followed by dilution into 1 M HEPES buffer to adjust the solution to physiological pH (7.4) for use during transfection. F Phe analog was supplied at concentrations ranging from 30 μM to 3 mM. For these experiments, the plasmid pair pAcBac1-sfGFP^{150TAG} 4×Ma-tRNA_{CUA} and pAcBac1-MaRS-F-Phe 4×Ma-tRNA_{CUA} was co-transfected in a 1:1 ratio. Prior to adding the DNA-reagent complexes, culture medium was replaced with fresh DMEM without FBS to minimize inhibition of transfection by secreted factors. DNA mixtures were prepared in 0.5 mL DMEM containing the indicated concentration of F Phe analog and added to the cells. Following transfection, cells were incubated for 24 or 48 h at 37° C. in a 5% CO₂ atmosphere to allow reporter expression, and mean fluorescence intensity (MFI) was measured by flow cytometry.

Sequence Listings
Prokaryotic tRNA
(SEQ ID NO: 1)
GGGGGACGGTCCGGCGACCAGCGGGTCTCTAAAACCTAGCATAGCGGGGT
TCGACACCCCGGTCTCTCGCCA
Eukaryotic tRNA
(SEQ ID NO: 2)
GGGGGACGGTCCGGCGACCAGCGGGTCTCTAAAACCTAGCATAGCGGGGT
TCGACACCCCGGTCTCTCG
RS 2345D2
(SEQ ID NO: 3)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLHLTDMGPRGDATEVLKNYISVVMKAAGLPDYDLVQ
EESDVYKETIDVEINGQEVCSAKVGPHYLDAAHDVHEPTSGAGFGLERLL
TIREKYSTVKKGGASISYLNGAKINSG
RS 2345E3
(SEQ ID NO: 4)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLHLSDMGPRGDATEVLKNYISVVMKAAGLPDYDLVQ
EESDVYKETIDVEINGQEVCSAKVGPHYLDAAHDVHEPTSGAGFGLERLL
TIREKYSTVKKGGASISYLNGAKINSG
RS 2345E4
(SEQ ID NO: 5)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNVYSVMRDLRDHTDGPVKIFEMGSCFR
KESHSGMHLEEFTMLHLQDMGPRGDATEVLKNYISVVMKAAGLPDYDLVQ
EESDVYKETIDVEINGQEVCSAGVGPHYLDAAHDVHEPWSGAGFGLERLL
TIREKYSTVKKGGASISYLNGAKINSG
RS 2345E6
(SEQ ID NO: 6)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNEYSVMRDLRDHTDGPVKIFEMGSCFR
KESHSGMHLEEFTMLHLQDMGPRGDATEVLKNYISVVMKAAGLPDYDLVQ
EESDVYKETIDVEINGQEVCSASVGPHYLDAAHDVHEPWSGAGFGLERLL
TIREKYSTVKKGGASISYLNGAKINSG
RS 236C9
(SEQ ID NO: 7)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLQLSDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 236E9
(SEQ ID NO: 8)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLALIDMGPRGDATEVLKNYISVVMKAAGLPDYDLVQ
EESDVYKETIDVEINGQEVCSAAVGPHYLDAAHDVHEPTSGAGFGLERLL
TIREKYSTVKKGGASISYLNGAKINSG
RS 236F7
(SEQ ID NO: 9)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNTYSVMRDLRDHTDGPVKIFEMGSCFR
KESHSGMHLEEFTMLTLIDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 236G11
(SEQ ID NO: 10)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLALCDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 236G7
(SEQ ID NO: 11)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLCLMDMGPRGDATEVLKNYISVVMKAAGLPDYDLVQ
EESDVYKETIDVEINGQEVCSAAVGPHYLDAAHDVHEPASGAGFGLERLL
TIREKYSTVKKGGASISYLNGAKINSG
RS 24B3
(SEQ ID NO: 12)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNHYSVMRDLRDHTDGPVKIFEMGSCFR
KESHSGMHLEEFTMLCLYDMGPRGDATEVLKNYISVVMKAAGLPDYDLVQ
EESDVYKETIDVEINGQEVCSASVGPHYLDAAHDVHEPWSGAGFGLERLL
TIREKYSTVKKGGASISYLNGAKINSG
RS 24D6
(SEQ ID NO: 13)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNAYSVMRDLRDHTDGPVKIFEMGSCFR
KESHSGMHLEEFTMLTLYDMGPRGDATEVLKNYISVVMKAAGLPDYDLVQ
EESDVYKETIDVEINGQEVCSAAVGPHYLDAAHDVHEPTSGAGFGLERLL
TIREKYSTVKKGGASISYLNGAKINSG
RS 24G1
(SEQ ID NO: 14)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLQLSDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 24G5
(SEQ ID NO: 15)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLHLHDMGPRGDATEVLKNYISVVMKAAGLPDYDLVQ
EESDVYKETIDVEINGQEVCSASVGPHYLDAAHDVHEPWSGAGFGLERLL
TIREKYSTVKKGGASISYLNGAKINSG
RS 25D8
(SEQ ID NO: 16)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNCYSVMRDLRDHTDGPVKIFEMGSCFR
KESHSGMHLEEFTMLHLQDMGPRGDATEVLKNYISVVMKAAGLPDYDLVQ
EESDVYKETIDVEINGQEVCSACVGPHYLDAAHDVHEPWSGAGFGLERLL
TIREKYSTVKKGGASISYLNGAKINSG
RS 25E7
(SEQ ID NO: 17)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLCLKDMGPRGDATEVLKNYISVVMKAAGLPDYDLVQ
EESDVYKETIDVEINGQEVCSATVGPHYLDAAHDVHEPWSGAGFGLERLL
TIREKYSTVKKGGASISYLNGAKINSG
RS 25F7
(SEQ ID NO: 18)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNHYSVMRDLRDHTDGPVKIFEMGSCFR
KESHSGMHLEEFTMLQLIDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 25H10
(SEQ ID NO: 19)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLALKDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 26B2
(SEQ ID NO: 20)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLALMDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 26B3
(SEQ ID NO: 21)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLALHDMGPRGDATEVLKNYISVVMKAAGLPDYDLVQ
EESDVYKETIDVEINGQEVCSAAVGPIPLDAAHDVHEPWSGAGFGLERLL
TIREKYSTVKKGGASISYLNGAKINSG
RS 26B4
(SEQ ID NO: 22)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLALQDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 26C6
(SEQ ID NO: 23)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLVLYDMGPRGDATEVLKNYISVVMKAAGLPDYDLVQ
EESDVYKETIDVEINGQEVCSAAVGPHYLDAAHDVHEPTSGAGFGLERLL
TIREKYSTVKKGGASISYLNGAKINSG
RS 26D5
(SEQ ID NO: 24)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLALFDMGPRGDATEVLKNYISVVMKAAGLPDYDLVQ
EESDVYKETIDVEINGQEVCSAGVGPHYLDAAHDVHEPWSGAGFGLERLL
TIREKYSTVKKGGASISYLNGAKINSG
RS 2B5
(SEQ ID NO: 25)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNQYSVMRDLRDHTDGPVKIFEMGSCFR
KESHSGMHLEEFTMLQLSDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 2C3
(SEQ ID NO: 26)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNAYSVMRDLRDHTDGPVKIFEMGSCFR
KESHSGMHLEEFTMLLLKDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 2C5
(SEQ ID NO: 27)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNVYSVMRDLRDHTDGPVKIFEMGSCFR
KESHSGMHLEEFTMLQLTDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 2D1
(SEQ ID NO: 28)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNAYSVMRDLRDHTDGPVKIFEMGSCFR
KESHSGMHLEEFTMLQLTDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 4A7
(SEQ ID NO: 29)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNMYSVMRDLRDHTDGPVKIFEMGSCFR
KESHSGMHLEEFTMLHLHDMGPRGDATEVLKNYISVVMKAAGLPDYDLVQ
EESDVYKETIDVEINGQEVCSAKVGPHYLDAAHDVHEPTSGAGFGLERLL
TIREKYSTVKKGGASISYLNGAKINSG
RS 4C8
(SEQ ID NO: 30)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLCLYDMGPRGDATEVLKNYISVVMKAAGLPDYDLVQ
EESDVYKETIDVEINGQEVCSAKVGPHYLDAAHDVHEPTSGAGFGLERLL
TIREKYSTVKKGGASISYLNGAKINSG
RS penB7
(SEQ ID NO: 31)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNFYSVMRDLRDHTDGPVKIFEMGSCFR
KESHSGMHLEEFTMLMLFDMGPRGDATEVLKNYISVVMKAAGLPDYDLVQ
EESDVYKETIDVEINGQEVCSAGVGPHYLDAAHDVHEPWSGAGFGLERLL
TIREKYSTVKKGGASISYLNGAKINSG
RS penF10
(SEQ ID NO: 32)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLVLKDMGPRGDATEVLKNYISVVMKAAGLPDYDLVQ
EESDVYKETIDVEINGQEVCSAGVGPHYLDAAHDVHEPWSGAGFGLERLL
TIREKYSTVKKGGASISYLNGAKINSG
RS PenF7
(SEQ ID NO: 33)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNVYSVMRDLRDHTDGPVKIFEMGSCFR
KESHSGMHLEEFTMLCLKDMGPRGDATEVLKNYISVVMKAAGLPDYDLVQ
EESDVYKETIDVEINGQEVCSAGVGPHYLDAAHDVHEPWSGAGFGLERLL
TIREKYSTVKKGGASISYLNGAKINSG
RS tpG2
(SEQ ID NO: 34)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNLYSVMRDLRDHTDGPVKIFEMGSCFR
KESHSGMHLEEFTMLQLTDMGPRGDATEVLKNYISVVMKAAGLPDYDLVQ
EESDVYKETIDVEINGQEVCSASVGPHYLDAAHDVHEPWSGAGFGLERLL
TIREKYSTVKKGGASISYLNGAKINSG
RS tpH6
(SEQ ID NO: 35)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNIYSVMRDLRDHTDGPVKIFEMGSCFR
KESHSGMHLEEFTMLQLVDMGPRGDATEVLKNYISVVMKAAGLPDYDLVQ
EESDVYKETIDVEINGQEVCSAAVGPHYLDAAHDVHEPWSGAGFGLERLL
TIREKYSTVKKGGASISYLNGAKINSG
RS 2345C3
(SEQ ID NO: 36)
MTVKYTDAQIQRLREYGNGTYEQKVFEDLASRDAAFSKEMSVASTDNEKK
IKGMIANPSRHGLTQLMNDIADALVAEGFIEVRTPIFISKDALARMTITE
DKPLFKQVFWIDEKRALRPMLAPNVYSVMRDLRDHTDGPVKIFEMGSCFR
KESHSGMHLEEFTMLHLQDMGPRGDATEVLKNYISVVMKAAGLPDYDLVQ
EESDVYKETIDVEINGQEVCSASVGPHYLDAAHDVHEPWSGAGFGLERLL
TIREKYSTVKKGGASISYLNGAKINSG

As used hereon, the term “about” refers to +5% of the specified value.

While illustrative embodiments have been illustrated and described, it will be appreciated that various changes can be made therein without departing from the spirit and scope of the invention.

The embodiments of the invention in which an exclusive property or privilege is claimed are defined as follows:

1. A pyrrolysine-based aminoacyl-tRNA synthetase competent to recognize a fluorinated phenylalanine analog and aminoacylate a tRNA, wherein the synthetase has an amino acid sequence of anyone of SEQ ID NO:3-36.

2. A pyrrolysine-based aminoacyl-tRNA synthetase/tRNA pair comprising a pyrrolysine-based aminoacyl-tRNA synthetase of claim 1 and a tRNA competent for site-specific encoding of a fluorinated phenylalanine analog within a protein.

3. The pyrrolysine-based aminoacyl-tRNA synthetase/tRNA pair of claim 2, wherein the DNA encoding the tRNA has SEQ ID NO: 1 or SEQ ID NO:2.

4. A method of site-specific encoding of a fluorinated phenylalanine analog in a cell, comprising transfecting/transforming/transducing a cell with a pyrrolysine-based aminoacyl-tRNA synthetase of claim 1 and a tRNA competent for site-specific encoding of a fluorinated phenylalanine analog within a protein to provide a transfected cell, and contacting the transfected cell with a fluorinated phenylalanine analog to provide the protein site-specifically encoded with a fluorinated phenylalanine analog.

5. The method of claim 4, wherein the cell is a prokaryotic cell.

6. The method of claim 4, wherein the cell is a eukaryotic cell.

7. The method of claim 4, wherein the fluorinated phenylalanine analog is selected from the group consisting of:
(a) 2-fluorophenylalanine;
(b) 4-fluorophenylalanine;
(c) 2,4-difluorophenylalanine;
(d) 2,5-difluorophenylalanine;
(e) 2,6-difluorophenylalanine;
(f) 2,3,6-trifluorophenylalanine;
(g) 2,3,4,5-tetrafluorophenylalanine; and
(h) pentafluorophenylalanine.

8. The method of claim 4, wherein the protein is a therapeutic protein.

9. An in cellulo method for genetically encoding a protein or a polypeptide of interest, comprising incorporating a fluorinated phenylalanine analog into a protein or a polypeptide by genetic encoding using the pyrrolysine-based aminoacyl-tRNA synthetase of claim 1 and a tRNA competent for site-specific encoding of a fluorinated phenylalanine analog within the protein or the polypeptide.

10. The method of claim 9, wherein the cell is a prokaryotic cell.

11. The method of claim 9, wherein the cell is a eukaryotic cell.

12. The method of claim 9, wherein the fluorinated phenylalanine analog is selected from the group consisting of:
(a) 2-fluorophenylalanine;
(b) 4-fluorophenylalanine;
(c) 2,4-difluorophenylalanine;
(d) 2,5-difluorophenylalanine;
(e) 2,6-difluorophenylalanine;
(f) 2,3,6-trifluorophenylalanine;
(g) 2,3,4,5-tetrafluorophenylalanine; and
(h) pentafluorophenylalanine.

13. The method of claim 9, wherein the protein or the polypeptide of interest is a therapeutic protein or therapeutic polypeptide.

14. A kit for in cellulo production of a fluorinated phenylalanine-labeled protein, comprising:
(a) DNA encoding a pyrrolysine-based aminoacyl-tRNA synthetase of claim 1 and a tRNA competent for site-specific encoding of a fluorinated phenylalanine analog; and
(b) a fluorinated phenylalanine analog.

15. A plasmid for facilitating in cellulo expression of a synthetase of claim 1 and its tRNA, comprising nucleic acid that encodes the expression of the synthetase and nucleic acid that encodes the expression its tRNA.

16. A cell, comprising nucleic acid that encodes the expression of the synthetase of claim 1 and nucleic acid that encodes the expression its tRNA.


SEQUENCE LISTING (36 sequences)

SEQ ID NO: 1 - DNA, 72 bp, synthetic construct
  source 1..72: mol_type = other DNA
gggggacggtccggcgaccagcgggtctctaaaacctagcatagcggggttcgacaccccggtctctcgcca

SEQ ID NO: 2 - DNA, 69 bp, synthetic construct
  source 1..69: mol_type = other DNA
gggggacggtccggcgaccagcgggtctctaaaacctagcatagcggggttcgacaccccggtctctcg

SEQ ID NO: 3 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLHLTDMGPRGDATEVLKNYISVVMKAAGLPDYDLVQEESDVYKETIDVEINGQEVCSAKVGPHYLDAAHDVHEPTSGAGFGLERLLTIREKYSTVKKGGASISYLNGAKINSG

SEQ ID NO: 4 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLHLSDMGPRGDATEVLKNYISVVMKAAGLPDYDLVQEESDVYKETIDVEINGQEVCSAKVGPHYLDAAHDVHEPTSGAGFGLERLLTIREKYSTVKKGGASISYLNGAKINSG

SEQ ID NO: 5 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNVYSVMRDLRDHTDGPVKIFEMGSCFRKESHSGMHLEEFTMLHLQDMGPRGDATEVLKNYISVVMKAAGLPDYDLVQEESDVYKETIDVEINGQEVCSAGVGPHYLDAAHDVHEPWSGAGFGLERLLTIREKYSTVKKGGASISYLNGAKINSG

SEQ ID NO: 6 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNEYSVMRDLRDHTDGPVKIFEMGSCFRKESHSGMHLEEFTMLHLQDMGPRGDATEVLKNYISVVMKAAGLPDYDLVQEESDVYKETIDVEINGQEVCSASVGPHYLDAAHDVHEPWSGAGFGLERLLTIREKYSTVKKGGASISYLNGAKINSG

SEQ ID NO: 7 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLQLSDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 8 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLALIDMGPRGDATEVLKNYISVVMKAAGLPDYDLVQEESDVYKETIDVEINGQEVCSAAVGPHYLDAAHDVHEPTSGAGFGLERLLTIREKYSTVKKGGASISYLNGAKINSG

SEQ ID NO: 9 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNTYSVMRDLRDHTDGPVKIFEMGSCFRKESHSGMHLEEFTMLTLIDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 10 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLALCDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 11 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLCLMDMGPRGDATEVLKNYISVVMKAAGLPDYDLVQEESDVYKETIDVEINGQEVCSAAVGPHYLDAAHDVHEPASGAGFGLERLLTIREKYSTVKKGGASISYLNGAKINSG

SEQ ID NO: 12 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNHYSVMRDLRDHTDGPVKIFEMGSCFRKESHSGMHLEEFTMLCLYDMGPRGDATEVLKNYISVVMKAAGLPDYDLVQEESDVYKETIDVEINGQEVCSASVGPHYLDAAHDVHEPWSGAGFGLERLLTIREKYSTVKKGGASISYLNGAKINSG

SEQ ID NO: 13 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNAYSVMRDLRDHTDGPVKIFEMGSCFRKESHSGMHLEEFTMLTLYDMGPRGDATEVLKNYISVVMKAAGLPDYDLVQEESDVYKETIDVEINGQEVCSAAVGPHYLDAAHDVHEPTSGAGFGLERLLTIREKYSTVKKGGASISYLNGAKINSG

SEQ ID NO: 14 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLQLSDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 15 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLHLHDMGPRGDATEVLKNYISVVMKAAGLPDYDLVQEESDVYKETIDVEINGQEVCSASVGPHYLDAAHDVHEPWSGAGFGLERLLTIREKYSTVKKGGASISYLNGAKINSG

SEQ ID NO: 16 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNCYSVMRDLRDHTDGPVKIFEMGSCFRKESHSGMHLEEFTMLHLQDMGPRGDATEVLKNYISVVMKAAGLPDYDLVQEESDVYKETIDVEINGQEVCSACVGPHYLDAAHDVHEPWSGAGFGLERLLTIREKYSTVKKGGASISYLNGAKINSG

SEQ ID NO: 17 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLCLKDMGPRGDATEVLKNYISVVMKAAGLPDYDLVQEESDVYKETIDVEINGQEVCSATVGPHYLDAAHDVHEPWSGAGFGLERLLTIREKYSTVKKGGASISYLNGAKINSG

SEQ ID NO: 18 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNHYSVMRDLRDHTDGPVKIFEMGSCFRKESHSGMHLEEFTMLQLIDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 19 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLALKDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 20 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLALMDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 21 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLALHDMGPRGDATEVLKNYISVVMKAAGLPDYDLVQEESDVYKETIDVEINGQEVCSAAVGPIPLDAAHDVHEPWSGAGFGLERLLTIREKYSTVKKGGASISYLNGAKINSG

SEQ ID NO: 22 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLALQDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 23 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLVLYDMGPRGDATEVLKNYISVVMKAAGLPDYDLVQEESDVYKETIDVEINGQEVCSAAVGPHYLDAAHDVHEPTSGAGFGLERLLTIREKYSTVKKGGASISYLNGAKINSG

SEQ ID NO: 24 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLALFDMGPRGDATEVLKNYISVVMKAAGLPDYDLVQEESDVYKETIDVEINGQEVCSAGVGPHYLDAAHDVHEPWSGAGFGLERLLTIREKYSTVKKGGASISYLNGAKINSG

SEQ ID NO: 25 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNQYSVMRDLRDHTDGPVKIFEMGSCFRKESHSGMHLEEFTMLQLSDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 26 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNAYSVMRDLRDHTDGPVKIFEMGSCFRKESHSGMHLEEFTMLLLKDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 27 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNVYSVMRDLRDHTDGPVKIFEMGSCFRKESHSGMHLEEFTMLQLTDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 28 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNAYSVMRDLRDHTDGPVKIFEMGSCFRKESHSGMHLEEFTMLQLTDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 29 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNMYSVMRDLRDHTDGPVKIFEMGSCFRKESHSGMHLEEFTMLHLHDMGPRGDATEVLKNYISVVMKAAGLPDYDLVQEESDVYKETIDVEINGQEVCSAKVGPHYLDAAHDVHEPTSGAGFGLERLLTIREKYSTVKKGGASISYLNGAKINSG

SEQ ID NO: 30 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLCLYDMGPRGDATEVLKNYISVVMKAAGLPDYDLVQEESDVYKETIDVEINGQEVCSAKVGPHYLDAAHDVHEPTSGAGFGLERLLTIREKYSTVKKGGASISYLNGAKINSG

SEQ ID NO: 31 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNFYSVMRDLRDHTDGPVKIFEMGSCFRKESHSGMHLEEFTMLMLFDMGPRGDATEVLKNYISVVMKAAGLPDYDLVQEESDVYKETIDVEINGQEVCSAGVGPHYLDAAHDVHEPWSGAGFGLERLLTIREKYSTVKKGGASISYLNGAKINSG

SEQ ID NO: 32 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLVLKDMGPRGDATEVLKNYISVVMKAAGLPDYDLVQEESDVYKETIDVEINGQEVCSAGVGPHYLDAAHDVHEPWSGAGFGLERLLTIREKYSTVKKGGASISYLNGAKINSG

SEQ ID NO: 33 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNVYSVMRDLRDHTDGPVKIFEMGSCFRKESHSGMHLEEFTMLCLKDMGPRGDATEVLKNYISVVMKAAGLPDYDLVQEESDVYKETIDVEINGQEVCSAGVGPHYLDAAHDVHEPWSGAGFGLERLLTIREKYSTVKKGGASISYLNGAKINSG

SEQ ID NO: 34 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLQLTDMGPRGDATEVLKNYISVVMKAAGLPDYDLVQEESDVYKETIDVEINGQEVCSASVGPHYLDAAHDVHEPWSGAGFGLERLLTIREKYSTVKKGGASISYLNGAKINSG

SEQ ID NO: 35 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNIYSVMRDLRDHTDGPVKIFEMGSCFRKESHSGMHLEEFTMLQLVDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG

SEQ ID NO: 36 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNVYSVMRDLRDHTDGPVKIFEMGSCFRKESHSGMHLEEFTMLHLQDMGPRGDATEVLKNYISVVMKAAGLPDYDLVQEESDVYKETIDVEINGQEVCSASVGPHYLDAAHDVHEPWSGAGFGLERLLTIREKYSTVKKGGASISYLNGAKINSG